(19) United States
(12) Patent Application Publication
(10) Pub. No.: US 2025/0228639 A1
(43) Pub. Date: Jul. 17, 2025

(54) STERILE DRAPE PREPARED FOR OR WITH AN MRI LOCAL COIL AS WELL AS PRODUCTION AND USE OF SUCH A STERILE DRAPE

(71) Applicant: Noras Holding GmbH & Co. KG, Höchberg (DE)

(72) Inventors: Manuel Noras, Würzburg (DE); Manuel Brückner, Gemünden am Main (DE)

(21) Appl. No.: 18/683,927

(22) PCT Filed: Sep. 26, 2023
(86) PCT No.: PCT/DE2023/100713

§ 371 Date: Feb. 15, 2024

(30) Foreign Application Priority Data
Sep. 28, 2022 (DE) 10 2022 125 038.5

(51) Int. Cl. A61B 46/23; A61B 46/00; A61B 46/20; G01R 33/34; G01R 33/341

(52) U.S. Cl. CPC A61B 46/23; A61B 46/40; A61B 2046/205; G01R 33/34007; G01R 33/341

(57) ABSTRACT

The present invention relates to a sterile drape for use in magnetic resonance tomographic examinations or operations using magnetic resonance tomographic imaging based on a patient drape of a known type, which is connected to a local coil of a magnetic resonance arrangement or prepared for such a connection by means of an area provided for this purpose is. The connection can be made at the factory according to a manufacturing process according to the invention or only immediately before the sterile drape is used as part of an examination or operation.

### CROSS-REFERENCE TO RELATED APPLICATIONS

This Application is a Section 371 National Stage Application of International Application No. PCT/DE2023/100713, filed Sep. 26, 2023, which is based on German Patent Application No. DE 10 2022 125 038.5, filed Sep. 28, 2022; the contents of each of which are hereby incorporated by reference in their entirety.

### FIELD

Embodiments of the present disclosure relate to a sterile drape for use in magnetic resonance imaging examinations or operations using magnetic resonance imaging prepared for connection or already connected at the factory to a local coil of a magnetic resonance arrangement, comprising a cloth made of sterile, flexible material, wherein the local coil can or is packaged sterile for the examination or operation. Embodiments of the present disclosure further relate to a manufacturing process for such sterile drapes.

### BACKGROUND

When carrying out medical examinations or operations that require the precise targeting or precise manipulation of a specific target volume, such as a focus of inflammation, or a tumor or other noticeable change, the examination or operation can be carried out using magnetic resonance imaging, by which the effect and the position of the instruments used in or relative to the target volume can be verified more or less in real time.

To record magnetic resonance imaging signals from the target volume, so-called local coils are used, which are placed in the immediate vicinity of the target area to be examined or operated on in order to be able to record the strongest possible signal. Since the operation or examination must be carried out under sterile conditions, the local coils, i.e. their surface, must be sterilized or the coil must be packaged sterilely. In the prior art, the latter is preferred because it is easier to accomplish.

For example, the local coils to be used during the examination or operation are wrapped directly before the operation with (pieces of) known sterile drapes used to cover the patient and glued to the cloth covering the patient with adhesive tape. However, this solution, which is still often used, is not satisfactory because a sufficiently sterile covering cannot always be guaranteed and wrapping a usually toroidal coil with the existing flat sterile drapes is difficult and sometimes only insufficiently successful. In addition, the preparation time required and the need for sterile personnel (i.e. people who have carried out the procedure prescribed before an operation to sterilize as much as possible all body and clothing surfaces that potentially come into contact with the patient) are high because wrapping and placing the coils is difficult to accomplish by just one sterile person alone.

More sophisticated solutions have therefore already been proposed in the prior art. For example, the US publication US 2015/0011870 A1 presents sterile covers made of flexible material for packaging magnetic resonance local coils, which are provided with an adhesive film on the inside, so that a local coil can be packaged sterile comparatively quickly by way of being placed on onto one half of the inside of the cover, covered with the other half and the two halves then being pressed together to create a germ-tight seal. The disadvantage of this solution, however, remains that the local coil packaged in such a sterile manner still has to be securely and permanently attached to a large sterile drape covering the patient at the actual place of use on the patient's body.

Although this disadvantage is overcome by the sterile drape shown in the patent U.S. Pat. No. 5,396,905, this comes at the price of other disadvantages. In the sterile drape proposed there, the local coil for the magnetic resonance imaging examination is already packed in a sterile manner at the factory between one of the known sterile drapes already mentioned for covering a large area of the patient and a cover glued to the cloth. It is also proposed to cover the local coil underneath this cover with a material that is difficult to cut and thus protect it from unwanted damage when carrying out surgical operations. The disadvantage of this sterile drape with integrated local coil proposed there is that the surgeon is limited to using a specific coil and cannot freely select it and adapt it to the examination immediately before carrying out the procedure or examination, for example depending on the size or incision to be made or the depth of the target point to be reached.

Against this background, the present invention has set itself the objective of finding a more flexible and easier to handle solution for the sterile application of an MRI local coil during an operation or examination.

This objective is solved by a sterile drape according to claim 1, which is prepared for the connection with a not necessarily sterile local coil, a sterile drape according to claim 10, which already comprises an integrated, sterilely packaged local coil and a method for producing such sterile drapes according to claim 12.

### SUMMARY

Embodiments of the present disclosure are based on the idea of providing the person carrying out the procedure or examination with the greatest possible freedom in terms of size and shape as well as the type of packaging of the local coil to be used. In addition, the preparation of the sterile drape covering the patient, i.e. the sterile packaging of the local coil, is made as simple and quick as possible.

In a first aspect, the invention presents two different solution variants, namely a first in which a sterile drape is provided, which is prepared for the sterile connection with a local coil, and a second in which the local coil is already connected to or integrated into the sterile drape. The first variant is presented in claims 1-8, claim 10 is directed to the second variant.

In one embodiment according to the first variant is at least one area prepared for the sterile connection of a coil. This is often located relatively centrally on the cloth, but can also be positioned in the edge regions of the cloth, so that even when positioned on the patient's extremities, for example on the head, arms or legs, a large-area covering also including the torso is still possible.

The sterile drape according to this first aspect essentially comprises a cloth made of sterile, flexible material as is generally well known for use in the operating room. In at least one place, this cloth has an area specially prepared for connection to a local coil, which can be implemented in various ways.

For one, the area can be a pocket made of sterile, flexible material that is sterile on the outside, for example the same material as the cloth or a transparent film. The local coil can be inserted into this pocket and closed sterilely.

Alternatively, the area can also consist of an adhesive surface to which the local coil itself can be stuck directly, or after it is packed in a sterile bag or embedded in a rigid, sterile and optionally easily sterilizable casing and then pressed onto the adhesive surface to securely bond it. In the folded, packaged state of the sterile drape according to some embodiments, the connection area designed as an adhesive surface is preferably covered with a protective film to prevent the sterile drape from unintentionally sticking to itself.

Embodiments of the second variant comprise a sterile drape in which the local coil is already firmly integrated. This can be done in such a way that the local coil is inserted into a pocket in the cloth, and the pocket is then glued or sewn shut. The local coil can also be embedded in a rigid, sterile covering, which in turn is permanently connected to the cloth, for example by gluing, welding, or sewing.

As a further possible embodiment, the coil can be packed sterilely by covering it with a flat cover, for example made of a textile material similar or identical to the sterile cloth itself, or a transparent film, and gluing, sewing, welding or is otherwise permanently connecting the cover to the cloth.

A further advantageous embodiment of a local coil that is already connected to the sterile drape is designed in such a way that the coil itself is glued directly to the cloth. In this case, the coil particularly preferably comprises an easily washable or sterilizable plastic covering of the conductive elements. The gluing or welding to the cloth made of sterile and flexible material of the sterile drape may take place via this plastic covering.

A second aspect of the present disclosure is a manufacturing method of a sterile drape according to one of the variants presented above, which is characterized by the four steps of providing a flexible sterile cloth, preparing the cloth for connection to a local coil, providing a local coil and connecting the local coil with the cloth. The preparation of the cloth for the connection of the local coil in the second step can be done by applying a pocket for inserting the local coil or applying an adhesive surface, whereby the adhesive surface also has the shape of a flat plastic element welded or sewn to the cloth, which on one side may have an adhesive film.

The advantages of the sterile drapes according to the first variant of the present disclosure are the flexibility in terms of size and shape of the local coil used, which can be selected and connected to the cloth up until shortly before the examination or operation is carried out. However, the sterile drape according to both variants has the advantage of being comparatively quick and easy to prepare for the examination: In the first variant, the preparation of the sterile drape is made possible by applying an area suitable for connecting a local coil, so that the immediately before the production of the cloth during the examination or operation can be carried out using the production process of the second aspect with only two people, only one of whom needs to be sterile. The sterile drape according to the second variant can even be used quickly and effectively by just one person, as it simply has to be removed from its sterile packaging and spread over the patient. This can be seen as a significant advantage and relief compared to the solutions known from the prior art.

Further advantageous embodiments can be found in the dependent claims and will be presented in detail below. It is obvious to the skilled person that, in principle, even if not expressly mentioned, they can be combined with one another, provided that they are not technically mutually exclusive.

In some embodiments of the sterile drape according to the first variant of the first aspect, the area of the drape prepared for connection to a local coil is a bag or pocket or pouch attached to one side of the drape and sterile at least on its outside made of a sterile, flexible material, in particular the same material as the drape or preferably a transparent film.

The local coil can be inserted into the bag pocket and the pocket can preferably be closed with a tab, the tab particularly preferably having an adhesive surface on an underside with which it can be glued to the cloth, so that the local coil is packaged completely sterile.

An alternative embodiment of the sterile drape according to the first aspect of the invention is the area of an adhesive surface prepared for connection to the local coil for attaching either the local coil itself, or a separate, sterile pouch in which the local coil is packed or one rigid, sterile or easily sterilizable casing in which the local coil is removably embedded or a flat cover made of a flexible material to cover the local coil in a germ-proof manner.

In embodiments with a rigid plastic casing for enveloping the local coil, this plastic casing is preferably formed of polyethylene ethylene ketone (PEEK) or polypropylene sulfone (PPSU).

In embodiments in which the local coil itself is glued directly to the cloth, it particularly preferably comprises a covering, in particular a plastic covering, of the conductive coil parts.

In the context of this disclosure, “outline” should only be understood as the external closed line surrounding a (three-dimensional) object or its projection onto a plane and, synonymously, for the area delimited by this line. On the other hand, “footprint” should refer to all closed lines that appear when the object is projected onto the plane or the inner surface enclosed between these lines. The outline and footprint are therefore only different if the object has holes or openings that are visible in the selected projection direction. For example, the outline of a torus-shaped object is a circular line/disc (or, depending on the direction of projection, an ellipse), but the footprint is an annulus.

In embodiments in which a flat cover made of sterile, flexible material is used for sterile packaging of the local coil, this cover particularly preferably has an outline which corresponds to the coil adhesive surface. In these embodiments, the cover can already be connected to the cloth at the factory, in particular in such a way that part of the edge of the cover coincides with an edge of the coil adhesive surface or is at least extends parallel to the edge of the coil adhesive surface at a distance that is small compared to the dimensions of the cover. For example, the adhesive surface and cover can have a rectangular outline and the cover can be connected to the cloth with one of the rectangle sides, this rectangle side coinciding with the corresponding side of the adhesive area or at least running close to it. The adhesive surface and/or the cover can, for example, have dimensions of 10×10 cm, 10×20 cm, 15×15 cm, 15×20 cm, 15×25 cm, 20×20 cm, or 20×30 cm.

In other embodiments with a flat cover made of textile material, this is a cover cloth that is similar to the cloth in terms of shape, size and/or material, which can differ from the cloth in that it has no adhesive surfaces or at least no coil adhesive surface.

The cloth and drape can each have an opening, in particular a circular one, for reaching through a sterilely packaged local coil. The openings are arranged in such a way that when the cloth and cover cloth of the same size and shape lie congruently on top of each other, the openings are concentric. The opening of the cover cloth is preferably slightly smaller than that of the cloth in order to ensure that the adhesive surface of the cloth is completely covered, especially taking into account that the cover cloth when placed over the local coil is slightly stretched and therefore the opening slightly enlarged when the cover cloth is attached to the coil adhesive surface.

The cloth with the (coil) adhesive surface for connection to the local coil preferably has a further (patient) adhesive surface on an opposite side to secure the cloth to the area of the patient to be examined.

Some embodiments of the present disclosure are directed to a kit comprising a cloth of the type described above prepared for connection to a local coil and a covering cloth without adhesive surfaces. As part of the manufacturing process according to the invention, the two cloths can be packed together at the factory, such as individually, sterilely packaged and delivered together in a package.

In some embodiments with an area in the form of a coil adhesive surface, this consists of a flat element integrated into the cloth, which may be made of plastic, which carries an adhesive film, in particular on its top side.

In some embodiments of the sterile drape with a coil adhesive surface, its outline corresponds to an enlarged outline of the local coil lying or projected on the drape.

In some embodiments of the sterile drape with an adhesive surface, this includes a coil area that is non-adhesive or at least poorly adhesive to the local coil, that is, depending on its material, the outline of which essentially corresponds to that of the local coil, so that the local coil itself is not or is hardly glued when it is placed on the adhesive area. This area can particularly preferably be formed by a protective film of the same layout as the local coil glued to the adhesive surface. In embodiments in which the adhesive surface as a whole is covered by a protective film, this large-area covering of the adhesive surface is glued over the coil area, in particular over and on top of a protective film having the layout of the local coil.

In the same embodiments, the one adhesive surface may leave out a central area, the size and shape of which corresponds to a reduced outline of a continuous opening of the local coil.

In some embodiments of the sterile drape according to the first variant of the first aspect of the invention, the area prepared for connection to the local coil is arranged on a side of the cloth facing away from the patient when the cloth is used as intended.

In some embodiments of the sterile drape according to the second aspect of the present invention, the local coil comprises a plastic covering which is connected to the cloth, in particular glued or welded or sewn.

In some embodiments of the sterile drape according to both variants of the first aspect of the invention or also a sterile drape produced according to the second aspect of the invention, the sterile drape has an opening that is concentric with the through-opening of the local coil.

In some embodiments of the manufacturing method of a sterile drape according to the first or second aspect of the invention, in further steps following the second step, the sterile drape prepared for connecting the local coil is folded and packaged in a sterile manner.

In some embodiments of the production of a sterile drape according to the first or second aspect of the invention, the steps of providing the local coil and connecting the local coil to the sterile drape made of flexible material are spatially and temporally separated from the first manufacturing steps of providing the sterile and flexible cloth and preparing it for connection to the coil. In particular, the local coil may only be connected immediately before use as part of a medical examination or operation.

In one embodiment of the production according to the invention, in the second step, a (coil) adhesive surface is applied to the cloth made of sterile, flexible material, which is covered with a non-adhesive protective film, and the sterile drape is folded and sterilely packaged in a subsequent step. In addition to the coil adhesive surface, a patient adhesive surface can be applied to the opposite side of the cloth, preferably concentric with the coil adhesive surface, and also covered with a cover film. The cloth can be provided in the area of the coil adhesive surface with an opening, in particular arranged concentrically in the coil adhesive surface, the shape of which corresponds to that of the opening of a local coil to be packaged sterilely and the size of which is slightly smaller than that of the opening of the local coil.

In some of these embodiments of the manufacturing method, in addition to the cloth, a cover cloth similar to the cloth in terms of material, shape and/or size without a coil adhesive surface is provided and is also folded and packaged sterilely, either together with the cloth or separately. The cloth and cover cloth can be used at corresponding points with an opening for reaching through a local coil that is sterilely packed between the cloth and cover cloth after carrying out the complete manufacturing process according to the invention and for this purpose has a shape corresponding to the shape of a local coil opening.

The opening in the cloth is smaller than the coil opening by an amount that ensures that the cover cloth can be securely glued to the circumferential strip of coil adhesive surface protruding into the opening of the coil placed concentrically on the coil adhesive surface. This strip can in particular have a width of 0.5-2.0 cm, in particular approximately 1 cm.

In order to compensate for the stretching that occurs during attachment, the opening in the cover cloth corresponding to the cloth opening may be slightly smaller than the cloth opening, in particular by approximately 1 mm to 20 mm, or between 1 and 10%, preferably 2-5 mm or 2-5%.

In some embodiments of the second aspect of the invention, in the last step of production, connecting the local coil to the sterile drape prepared for connecting the coil comprises the following sub-steps: The packaging of the sterile drape is opened by a person who does not have to be sterile him/herself. The sterile drape is then removed from the packaging and unfolded by a sterile person. The local coil, which is not necessarily sterile, is then placed on the adhesive surface, which can be done by a person who is not necessarily sterile, and is preferably done by the same person who opened the packaging. The local coil is positioned, in particular, on the adhesive surface in an existing coil area with which it is difficult or impossible to bond to the coil. In a final step, the coil is covered and sealed in a germ-proof manner by placing a cover on it and gluing the cover to the sterile, flexible cloth by pressing it over the entire surface onto the adhesive surface. This last step must be carried out by a sterile person, as the outside or top side of the cover that potentially comes into contact with the patient must be touched. This sterile person is preferably the same person who had already removed and unfolded the sterile drape for packaging in the second sub-step. Thus, the completion of the production of a sterile drape according to the invention and its preparation for a medical examination using magnetic resonance imaging can be accomplished quickly and easily by just two people, only one of whom has to be sterile.

Further advantages, properties, and features of the embodiments of the present disclosure are apparent from the following exemplary embodiments explained with reference to the figures. These are only intended to explain the invention and not limit it in any way.

### BRIEF DESCRIPTION OF THE DRAWINGS

It is shown in:

FIG. 1: A representation of a sterile drape prepared to hold a local coil according to a first embodiment of the invention.

FIG. 2: A representation of a sterile drape prepared for connection to a local coil according to a second and third embodiment of the invention.

FIG. 3: A representation of a sterile drape prepared for connection to a local coil according to a fourth embodiment of the invention.

FIG. 4A: A representation of a sterile drape prepared for connection to a local coil according to a fifth embodiment of the invention.

FIG. 4B: A detailed representation of the area of the sterile drape from FIG. 4A prepared for connection to a local coil.

FIG. 5A: A perspective view of a fifth embodiment of a sterile drape according to the invention comprising a cloth with a coil adhesive area and a drape.

FIG. 5B: A schematic section through the embodiment of FIG. 5A.

### DETAILED DESCRIPTION OF ILLUSTRATIVE EMBODIMENTS

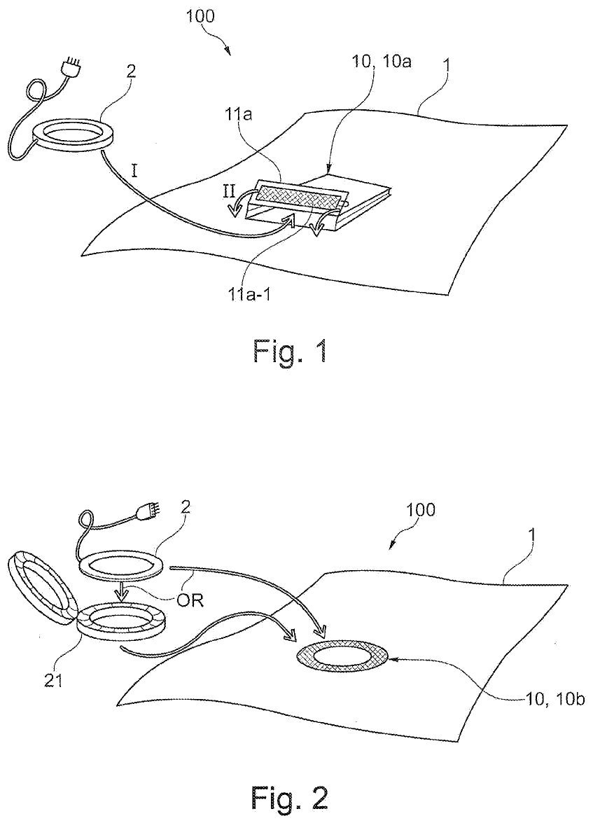

FIG. 1 shows a perspective view, not true to scale, of a sterile drape according to a first embodiment. The sterile drape 100 comprises a cloth 1 made of a sterile, flexible material, such as a textile patient drape, as is well known in the art. This cloth 1 has an area 10 prepared for connection to a local coil 2 of a magnetic resonance tomography arrangement in the form of a pocket 10a into which the local coil 2 can be inserted, as indicated by the double arrow I. After the coil 2 has been completely pushed in, the opening of the pocket 10a can be closed in a germ-tight manner by means of the tab 11a, indicated in the figure by the double arrows II. For this purpose, the tab 11a has an adhesive film 11a-1 on the underside. An adhesive film, not shown, can be attached inside the bag, by means of which the material of the bag 10a can be glued to the cloth 1 through the opening of the local coil.

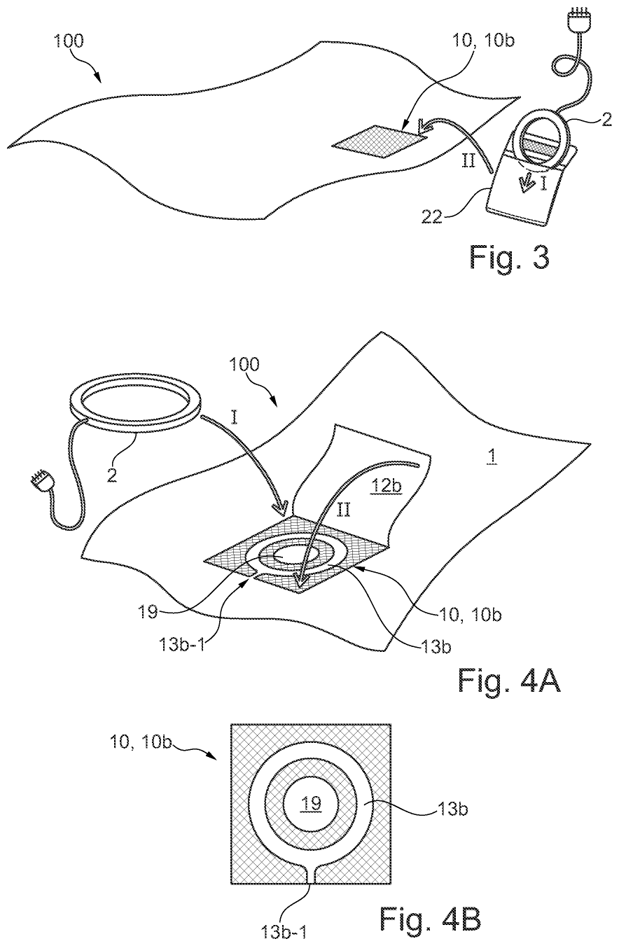

FIG. 2 shows a perspective view, not true to scale, of two further embodiments of a sterile drape according to the invention. What they have in common with the first embodiment is the flexible, sterile, in particular textile, cloth 1. However, in these embodiments, the area 10 present on the cloth 1 for preparation with the local coil is an annular adhesive region 10b. According to a second embodiment, the local coil 2 can be glued directly onto this, as indicated by the upper double arrow. Alternatively, in a third embodiment, the local coil 2 can also be embedded in a rigid, germ-proof, preferably easily washable or sterilizable plastic casing 21 and this plastic casing is then glued to the adhesive area 10b. The order of the steps of embedding the local coil 2 and gluing on the plastic casing 21 is arbitrary. FIG. 3 shows a further not-to-scale perspective view of a sterile drape according to the invention according to a fourth embodiment. In this embodiment, the cloth 1, which is already known from the previously described embodiments, has a rectangular adhesive area 10b onto which the local coil 2 inserted into the sterile bag 22 can be glued.

FIGS. 4A and 4B show different views of a fifth embodiment of a sterile drape according to the invention, FIG. 4A shows a perspective view of the sterile drape as a whole, FIG. 4B shows a top view of the area 10 of the cloth 1. The area 10 prepared for connection to the local coil 2 of the flexible, sterile cloth 1 is an adhesive area 10b with a rectangular, for example square, outline as specifically shown and particularly visible in FIG. 4B. In the adhesive area 10b, an annular coil area 13b is left out, on which the local coil 2 is preferably positioned. This prevents the coil 2 itself from sticking to the cloth 1, which simplifies and speeds up the removal of the coil after the examination or operation. A cover 12b, the outline of which essentially corresponds in shape and dimension to the adhesive area 10b, is attached to the cloth 1 in such a way that an edge of the cover 12b coincides with an edge of the adhesive area 10b. However, the cover 12b is preferably made of sufficiently flexible material and/or is dimensioned slightly larger than the adhesive area in order to compensate for the shortening when the local coil 2, which has a finite height, is covered. As indicated by the double arrows I and II, in the context of the production according to the invention of a sterile drape with a local coil from the sterile drape 100 prepared for preparation with the local coil 2 and the local coil, in a first step I the coil is placed on the coil area 13b of the adhesive area 10b positioned and then the cover 12b is folded over the coil. The cover 12b is then pressed flat onto the adhesive area 10b in order to ensure complete sterile enclosure of the local coil 2. A cable area 13b-1 of the coil area 13-b can be present to accommodate a data cable of the local coil.

The cloth 1 according to this embodiment has an opening 19. When connected to the local coil 2, this opening 19 is concentric with the opening of the local coil 2.

In the figures described above, a sterile drape according to the first variant of the first aspect of the invention was presented, in which the local coil is connected to the sterile drape specifically prepared for this purpose immediately before use as part of an examination or operation. In principle, all of the above-described embodiments can also be provided with a sterilely packaged local coil at the factory according to the second variant of the first aspect of the invention.

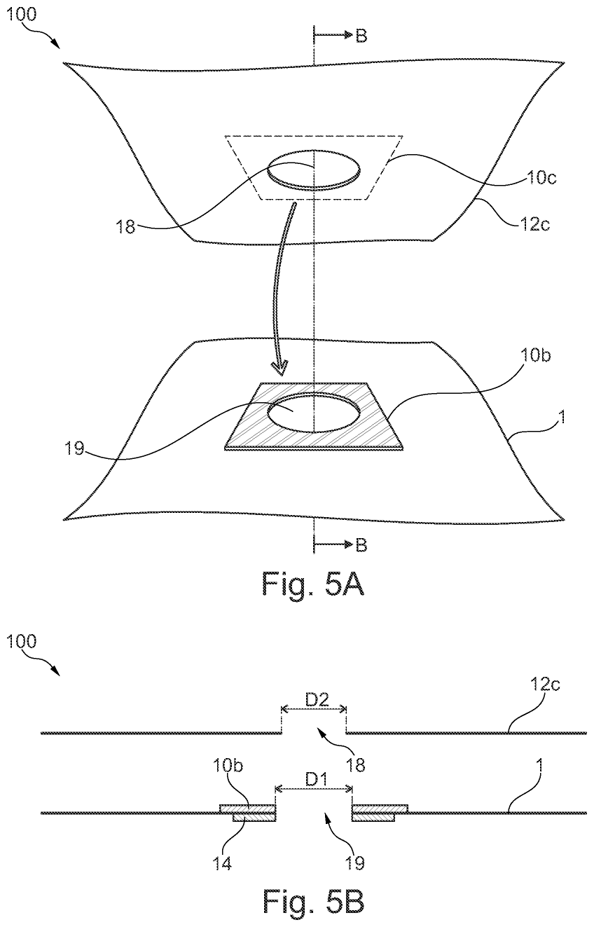

FIGS. 5A and 5B show two views of a further preferred embodiment of the invention.

The sterile drape 100 of this embodiment comprises a cloth 1 with a coil adhesive area 10b on an upper side and a circular opening 19 arranged in the middle therein. As can be seen in the schematic section of FIG. 5B, on the side opposite the coil adhesive area, there is provided the patient adhesive area 14 surrounding the opening 19, which serves to fix the sterile drape 100 to the area of the patient to be examined. The Coil adhesive area 10b and the patient adhesive area 14 can be of different sizes, as shown. Alternatively, they are the same size and otherwise designed in the same way, so that they can both serve as a coil and patient adhesive area. This has the advantage that users do not have to pay attention to the orientation of the cloth 1 when they spread the cloth 1 over the patient.

For covering and sterilely packaging a local coil (not shown), the present embodiment of the sterile drape 100 includes the cover cloth 12c, which, as shown, is chosen to be the same as the cloth 1 in terms of its size and shape, but also in terms of the (textile) material can. The covering cloth 1 has a likewise circular opening 18 at a position corresponding to the position of the opening 19 in the cloth 1, which, however, does not have to be surrounded by an adhesive area. The diameter D2 of the opening 18 of the drape 12c is preferably chosen to be slightly smaller than the diameter D1 of the opening 19 of the cloth 1, as can be seen from FIG. 5B.

For the sterile packaging of the local coil, when carrying out the manufacturing method according to the invention in such a way that the connection to the local coil is spatially and/or temporally separated from the provision and preparation of the cloth 1, the coil is positioned on the coil adhesive area of the cloth 1 by a person who is not necessarily sterile, advantageously in such a way that an opening of the local coil comes to lie concentrically with the opening 19 of the cloth 1. Subsequently, as indicated by the double arrow in FIG. 5A, the cover cloth 12c is placed onto and at least partially attached (where this is not prevented by the local coil in between) by a sterile person in order not to endanger the external sterility of the sterile drape 100. It is also preferable to ensure that the corners of the cover cloth 12c lie on those of the cloth 1. However, this is not absolutely necessary.

The smaller diameter D2 of the cover cloth opening 18 compared to the diameter D1 of the cloth opening 19 ensures that despite the stretching that occurs when the area 10c of the cover cloth 12c is attached to the coil adhesive area 10b of the cloth 1 due to the thickness of the local coil, no part of the coil adhesive area 10b is exposed by the opening 18 of the cover cloth 12c.

Although the embodiments of the present disclosure have been described with reference to preferred embodiments, workers skilled in the art will recognize that changes may be made in form and detail without departing from the spirit and scope of the present disclosure.

### List of Reference Signs

100 sterile drape
1 cloth
10 prepared area
10a pocket
11a tab
11a-1 adhesive film
10b coil adhesive area
10c area of 12c, which comes to lie on 10b
12b covering
12c cover cloth
13b coil area
13b-1 cable area
14 patient adhesive area
18 opening in 12c
19 opening in 1
D1 diameter of 19
D2 diameter of 18
2 local coil
21 rigid plastic casing
22 flexible sterile pouch

1. A sterile drape for use in magnetic resonance imaging examinations or operations by magnetic resonance imaging using a magnetic resonance arrangement having a local coil, the sterile drape comprising:
a cloth made of sterile and flexible material,
wherein the cloth includes a region that is prepared for connection to the local coil.

2. The sterile drape according to claim 1, wherein the region includes a bag having a sterile exterior and is made of sterile and flexible material and/or a transparent film, wherein the bag is configured to contain the local coil.

3. The sterile drape according to claim 2, wherein the bag includes a tab comprising an adhesive strip on an underside of the tab that faces the cloth, wherein the tab is configured to close an opening to the bag.

4. The sterile drape according to claim 1, wherein the region includes a coil adhesive surface configured to attach one of the following items to the cloth:
the local coil,
sterile pouch configured to contain the local coil,
a rigid, sterile or sterilizable casing, in which the local coil is embedded, made of plastic, polyether ether ketone (PEEK) or polyphenylsulfone (PPSU), or
a cover covering the local coil in a germ-proof manner, the cover made of a sterile and flexible material, a film, and/or a transparent film.

5. The sterile drape according to claim 4, further comprising the cover, wherein the cover:
has an outline that corresponds to an outline of the coil adhesive surface,
includes an edge that is connected to the cloth and coincides with an edge of the coil adhesive surface or extends parallel to the edge of the coil adhesive surface, and/or
comprises the sterile and flexible material of the cloth.

6. The sterile drape according to claim 5, wherein the cloth has a cloth opening arranged within the coil adhesive surface and the cover has a similarly shaped cover opening aligned with the cloth opening.

7. The sterile drape according to claim 6, wherein the cover opening and the cloth opening are circular, and the cover opening has a diameter that is smaller than a diameter of the cloth opening by 0.5 cm to 2 cm or by 1-% to 10-%.

8. The sterile drape according to claim 4, wherein the coil adhesive surface:
is a flat element made of rigid plastic integrated into the cloth,
corresponds to an enlarged outline of the local coil lying or projected on the cloth,
is rectangular or square, and/or
has a coil area configured to have a reduced adhesion to the local coil relative to a remainder of the coil adhesive surface, an outline of the coil area corresponds to an outline of the local coil, wherein the coil area is formed from a protective film of the outline of the local coil and is glued to a continuous adhesive surface.

9. The sterile drape according to claim 4, further comprising a patient adhesive surface on a side of the cloth that is opposite the coil adhesive surface, the patient adhesive surface being arranged concentrically or congruent with the coil adhesive surface.

10. A sterile drape for use in magnetic resonance imaging examinations and operations by magnetic resonance imaging using a magnetic resonance arrangement, the sterile drape comprising:
a cloth made of sterile and flexible material,
a local coil of the magnetic resonance arrangement,
wherein the local coil:
is inserted into a pocket of the cloth having an opening that is closed by a tab,
is embedded in a rigid, sterile casing that is connected to the cloth,
is covered in a sterile manner with a cover made of sterile and flexible material attached to a coil adhesive surface of the cloth, or
includes a sterile plastic covering that is glued or welded to the cloth.

11. The sterile drape according to claim 10, wherein the cloth has an opening that is concentric with a continuous opening of the local coil.

12. A method of producing of a sterile drape for a magnetic resonance imaging examination or operation on a patient using a magnetic resonance arrangement, the method comprising steps of:
a. providing a flexible and sterile cloth,
b. preparing the cloth for connection to a local coil of the magnetic resonance arrangement including:
forming a pocket on the cloth for inserting the local coil, the pocket being made of flexible and sterile material or a transparent film, and/or
applying a coil adhesive surface to the cloth
c. providing the local coil, and
d. connecting the local coil to the cloth by:
inserting the local coil into the pocket, or
attaching the local coil to the coil adhesive surface.

13. The method according to claim 12, wherein:
following step b, the method comprises folding and sterilely packaging the cloth in a package, and/or
steps c and d are performed at a place of use of the sterile drape with the magnetic resonance arrangement and are spatially and/or temporally separated from steps a and b, which are carried out at a factory.

14. The method according to claim 13, wherein:
steps c and d are carried out spatially and/or temporally separated from steps a and b,
the coil adhesive surface in step b is covered with a non adhesive film,
connecting the local coil to the cloth comprises removing the non adhesive film from the coil adhesive surface and attaching the local coil to the coil adhesive surface, and
the method includes covering the local coil with a cover.

15. The method according to claim 13, wherein:
step b comprises applying the coil adhesive surface to the cloth and covering the coil adhesive surface with a non-adhesive protective film, and
step d comprises the following steps that are carried out by a first sterile person and a second person:
i. opening the package containing the sterile drape by the second person without touching the sterile drape,
ii. removing and unfolding the sterile drape and removing the protective film by the first person,
iii. setting the unfolded sterile drape down,
iv. placing the local coil on the coil area by the second person, and
v. attaching a cover to the sterile drape by the first person including pressing the cover onto the coil adhesive area to enclose the local coil in germ-tight manner between the cover and the cloth.
the coil adhesive surface includes a coil area configured to have a reduced adhesion to the local coil than a remainder of the coil adhesive surface, and

16. The method according to claim 15, wherein:
the sterile drape includes a patient adhesive surface on a side of the cloth that is opposite the coil adhesive surface, and
the method includes:
placing the unfolded sterile drape on a patient with the coil adhesive surface facing upwards in such a way that the coil adhesive surface and an opening in the coil adhesive surface is placed on an area to be examined or operated on in step iii, or
placing a first side of the cover on a sterile surface, placing the local coil on a second side of the cover that is opposite the first side with an opening of the coil being concentric to an opening in the cover, and, in steps iii and iv, setting the sterile drape down on the cover with the coil adhesive surface above the local coil and with an opening in the cloth being concentric to the opening of the local coil.
